SEQUENCE LISTING (52 sequences)

SEQ ID NO: 1 - protein, 141 aa, Mus musculus
  Mouse Nix-AF067395
MSHVHNNNNNCGAGNSSWVMNSSNGNNGNGKNGGHVSSSSHNGDMKDAHSGSSSRGSSHCDSSDGMDVMHTSRDHSSSVVGKVAKKSADWVSDWSSRNKHRHKRAASSMRKSGAMKKGGSAKVSSHVAGGYGKRSTSASTY

SEQ ID NO: 2 - protein, 219 aa, Homo sapiens
  Human Nix-4138826
MSSHLVEPPPPLHNNNNNCEENEQSLPPPAGLNSSWVELPMNSSNGNDNGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRSVSLSMRKSGAMKKGGIFSAEFLKVFIPSLFLSHVLALGLGIYIGKRLSTPSASTY

SEQ ID NO: 3 - protein, 206 aa, Mus musculus
  Mouse Nix y2H
NNNNNCEEGEQPLPPPAGLNSSWVELPMNSSNGNENGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRAASLSMRKSGAMKKGGIFSAEFLKVFIPSLFLSHVLALGLGIYIGKRLSTPSASTY

SEQ ID NO: 4 - protein, 175 aa, Mus musculus
  Mouse Siva-AF033112
MPKRSCPFADAAPLQLKVHVGLKELSHGVFAERYSREVFERTKQLLFQGARAYRDHISSEDCSVNHLQESLKSGVVGAPQPARGQMLIGPDGRLTRCQAQASEGGLPRTAPIACSSCMRSVDGKAVCSQCERALCGQCVYTSWGCGALACVLCGLADYADDGEKTLCTSCAMFEA

SEQ ID NO: 5 - protein, 124 aa, Homo sapiens [sequence omitted]

SEQ ID NO: 6 - protein, 197 aa, Mus musculus
  Mouse Siva y2H
FGSEAKNSARGLGARRAARPATMPKRSCPFADAAPLQLKVHVGLKELSHGVFAERYSREVFERTKQLLFQGARAYRDHISSEDCSVNHLQESLKSGVVGAPQPARGQMLIGPDGRLTRCQAQASEGGLPRTAPIACSSCMRSVDGKAVCSQCERALCGQCVYTSWGCGALACVLCGLADYADDGEKTLCTSCAMFEA

SEQ ID NO: 7 - protein, 490 aa, Mus musculus
  Mouse Mytl kinase-AF175892
MTMPTEGTPPPLSGTPIPVPAYFRHAEPGFSLKRPGGLSRSLPPRPPAKGCIPVSRLFPPRTPGWHQPQPRRVSFLCETSEPLQSPGYDPSRPESFFQQNFQRLSRLGHGSYGEVFKVRSKEDGRLYAVKRYMSPFRGPKDRTRKLAEVGGHEKVGQHPHCVRLERAWEEGGILYLQTELCGPSLQQHCEAWGASLPEAQVWGYLRDILLALDHLHSQGLVHLDVKPANIFLGPRGRCKLGDFGLLVELGSTGAGEAQEGDPRYMAPELLQGSYGTAADVFSLGLTILEVACNMELPHGGEGWQQLRQGYLPPEFTAGLSSELRSVLAMMLEPDPQLRATAEALLALPMLRQPRPWNVLWYMAAEALSRGWALWQALVTLLCWLWHGLVHPASWLQPPGPPATPLGSPPCSPLMNSTLSSSWDNDSIGPSLSPETVLSRITRRTSTPRGRYIPRDALDLTDVDSEPPRGPCPTFEPRNLLSLFEDSLDPA

SEQ ID NO: 8 - protein, 499 aa, Homo sapiens
  Human Myt1 kinase-2914674
MLERPPALAMPMPTEGTPPPLSGTPIPVPAYFRHAEPGFSLKRPRGLSRSLPPPPPAKGSIPISRLFPPRTPGWHQLQPRRVSFRGEASETLQSPGYDPSRPESFFQQSFQRLSRLGHGSYGEVFKVRSKEDGRLYAVKRSMSPFRGPKDRARKLAEVGSHEKVGQHPCCVRLEQAWEEGGILYLQTELCGPSLQQHCEAWGASLPEAQVWGYLRDTLLALAHLHSQGLVHLDVKPANIFLGPRGRCKLGDFGLLVELGTAGAGEVQEGDPRYMAPELLQGSYGTAADVFSLGLTILEVACNMELPHGGEGWQQLRQGYLPPEFTAGLSSELRSVLVMMLEPDPKLRATAEALLALPVLRQPRAWGVLWCMAAEALSRGWALWQALLALLCWLWHGLAHPASWLQPLGPPATPPGSPPCSLLLDSSLSSNWDDDSLGPSLSPEAVLARTVGSTSTPRSRCTPRDALDLSDINSEPPRGSFPSFEPRNLLSLFEDTLDPT

SEQ ID NO: 9 - protein, 150 aa, Mus musculus
  Mouse kinase y2h
AEALLALPMLRQPRPWNVLWYMAAEALSRGWALWQALVTLLCWLWHGLVHPASWLQPPGPPATPLGSPPCSPLMNSTLSSSWDNDSIGPSLSPETVLSRITRRTSTPRGRYIPRDALDLTDVDSEPPRGPCPTFEPRNLLSLFEDSLDPA

SEQ ID NO: 10 - protein, 178 aa, Mus musculus
  Mouse Cyclin A2-X75483
MPGTSRHSGRDAGSALLSLHQEDQENVNPEKLAPAQQPRAQAVLKAGNVRGPAPQQKLKTRRVAPLKDLPINDEHVTAGPSWKAVSKQPAFTIHVDEAEETQKRPAELKETECEDALAFNAAVSLPAARKPLTPLDYPMDGSFESPHAMDMSIVLEDKPVNVNEVPDYQEDIHTYLRE

SEQ ID NO: 11 - protein, 244 aa, Homo sapiens
  Human Cyclin A2-510604
MEVKCKPKVGYMKRQPDITNSMRAILVDWLVEVGEEYKLQNETLHLAVNYIDRFLSSMSVLRGKLQLVGTAAMLLASKFEEIYPPEVAEFVYITDDTYSKKQVLRMEHLVLKVLAFDLAAPTVNQFLTQYFLHLQPANCKVESLAMFLGELSLIDADPYLKYLPSLIAGAAFHLALYTVTGQSWPESLAQQTGYTLESLKPCLVDLHQTYLKPPQHAQQSIREKYKHSKYHSVSLLNPPETLSV

SEQ ID NO: 12 - protein, 75 aa, Mus musculus
  Mouse Cyclin A2 y2H
AAFHLALYTVTGQSWPESLAQQTGYTLESLKPCLVDLHQTYLKPPQHAQQSIREKYKHSKYHSVSLLNPPETLSV

SEQ ID NO: 13 - protein, 438 aa, Mus musculus
  Mouse PM-SCL-9506980
MKETPLSNCERRFLLRAIEEKKRLDGRQTYDYRNIRISFGTDYGCCIVELGKTRVLGQVSCELVSPKLNRATEGILFFNLELSQMAAPAFEPGRQSDLLVKLNRLLERCLRNSKCIDTESLCVVAGEKVWQIRVDLHLLNHDGNIIDAASIAAIVALCHFRRPDVSVQGEEVTLYTPEERDPVPLSIHHMPICVSFAFFQQGTYLLVDPNEREERVMDGLLVIAMNKHREICTIQSSGGIMLLKDQVFRCSKIAGVKVAEITELIQKALENDQRVRKEGGKFGFAESIANQRITAFKMETAPIDTSNIEERAEEIIAEAEPPPEVVSQPVLWTPGTAQIGDGIENSWGDLEDSEKEEEEEEGGIDEAVILDDTKMDTGEVSDIGSQGAPIVLSDSEEEEMIILEPEKNPKKIRAQTSANQKAPSKGQGKRKKKKRTAN

SEQ ID NO: 14 - protein, 104 aa, Mus musculus
  Mouse PM-SCL y2h
GTAQIGDGIENSWGDLEDSEKEEEEEEGGIDEAVILDDTKMDTGEVSDIGSQGAPIVLSDSEEEEMIILEPEKNPKKIRAQTSANQKAPSKGQGKRKKKKRTAN

SEQ ID NO: 15 - protein, 255 aa, Mus musculus
  Mouse C8 proteasome-NM_011181
MSSIGTGYDLSASTFSPDGRVFQVEYAMKAVENSSTAIGIRCKDGVVFGVEKLVLSKLYEEGSNKRLFNVDRHVGMAVAGLLADARSLADIAREEASNFRSNFGYNIPLKHLADRVAMYVHAYTLYSAVRPFGCSFMLGSYSANDGAQLYMIDPSGVSYGYWGCAIGKARQAAKTEIEKLQMKEMTCRDVVKEVAKIIYIVHDEVKDKAFELELSWVGELTKGRHEIVPKDIREEAEKYAKESLKEEDESDDDNM

SEQ ID NO: 16 - protein, 251 aa, Mus musculus
  Mouse C8 proteasome
GTGYDLSASTFSPDGRVFQVEYAMKAVENSSTAIGIRCKDGVVFGVEKLVLSKLYEEGSNKRLFNVDRHVGMAVAGLLADARSLADIAREEASNFRSNFGYNIPLKHLADRVAMYVHAYTLYSAVRPFGCSFMLGSYSANDGAQLYMIDPSGVSYGYWGCAIGKARQAAKTEIEKLQMKEMTCRDVVKEVAKIIYIVHDEVKDKAFELELSWVGELTKGRHEIVPKDIREEAEKYAKESLKEEDESDDDNM

SEQ ID NO: 17 - protein, 1071 aa, Homo sapiens
  Human CRM1-2626839
MPAIMTMLADHAARQLLDFSQKLDINLLDNVVNCLYHGEGAQQRMAQEVLTHLKEHPDAWTRVDTILEFSQNMNTKYYGLQILENVIKTRWKILPRNQCEGIKKYVVGLIIKTSSDPTCVEKEKVYIGKLNMILVQILKQEWPKHWPTFISDIVGASRTSESLCQNNMVILKLLSEEVFDFSSGQITQVKSKHLKDSMCNEFSQIFQLCQFVMENSQNAPLVHATLETLLRFLNWIPLGYIFETKLISTLIYKFLNVPMFRNVSLKCLTEIAGVSVSQYEEQFVTLFTLTMMQLKQMLPLNTNIRLAYSNGKDDEQNFIQNLSLFLCTFLKEHDQLIEKRLNLRETLMEALHYMLLVSEVEETEIFKICLEYWNHLAAELYRESPFSTSASPLLSGSQHFDVPPRRQLYLPMLFKVRLLMVSRMAKPEEVLVVENDQGEVVREFMKDTDSINLYKNMRETLVYLTHLDYVDTERIMTEKLHNQVNGTEWSWKNLNTLCWAIGSISGAMHEEDEKRFLVTVIKDLLGLCEQKRGKDNKAIIASNIMYIVGQYPRFLRAHWKFLKTVVNKLFEFMHETHDGVQDMACDTFIKIAQKCRRHFVQVQVGEVMPFIDEILNNINTIICDLQPQQVHTFYEAVGYMIGAQTDQTVQEHLIEKYMLLPNQVWDSIIQQATKNVDILKDPETVKQLGSILKTNVRACKAVGHPFVIQLGRIYLDMLNVYKCLSENISAAIQANGEMVTKQPLIRSMRTVKRETLKLISGWVSRSNDPQMVAENFVPPLLDAVLIDYQRNVPAAREPEVLSTMAIIVNKLGGHITAEIPQIFDAVFECTLNMINKDFEEYPEHRTNFFLLLQAVNSHCFPAFLAIPPTQFKLVLDSIIWAFKHTMRNVADTGLQILFTLLQNVAQEEAAAQSFYQTYFCDILQHIFSVVTDTSHTAGLTMHASILAYMFNLVEEGKISTSLNPGNPVNNQIFLQEYVANLLKSAFPHLQDAQVKLFVTGLFSLNQDIPAFKEHLRDFLVQIKEFAGEDTSDLFLEEREIALRQADEEKHKRQMSVPGIFNPHEIPEEMCD

SEQ ID NO: 18 - protein, 236 aa, Mus musculus
  Mouse CRM1 y2H
ARGGEVMPFIDEILNNINTIICDLQPQQVHTFYEAVGYMIGAQTDQTVQEHLIEKYMLLPNQVWDSIIQQATKNVDILKDPETVKQLGSILKTNVRACKAVGHPFVIQLGRIYLDMLNVYKCLSENISAAIQANGEMVTKQPLIRSMRTVKRETLKLISGWVSRSNDPQMVAENFVPPLLDAVLIDYQRNVPAAREPEVLSTMAIIVNKLGGHITAEIPQIFDAVFECTLNMINKD

SEQ ID NO: 19 - protein, 172 aa, Mus musculus
  Mouse TCTP NP_033455
MIIYRDLISHDELFSDIYKIREIADGLCLEVEGKMVSRTEGAIDDSLIGGNASAEGPEGEGTESTVVTGVDIVMNHHLQETSFTKEAYKKYIKDYMKSLKGKLEEQKPERVKPFMTGAAEQIKHILANFNNYQFFIGENMNPDGMVALLDYREDGVTPFMIFFKDGLEMEKC

SEQ ID NO: 20 - protein, 172 aa, Homo sapiens; identical to SEQ ID NO: 19
  Human TCTP- NP_003286

SEQ ID NO: 21 - protein, 34 aa, Mus musculus
  Mouse TCTP y2H
NMNPDGMVALLDYREDGVTPFMIFFKDGLEMEKC

SEQ ID NO: 22 - protein, 204 aa, Mus musculus
  Tetraspan Net6 (SAS)- AF120265
MVCGGFACSKNCLCALNLLYTLVSLLLIGIAAWGIGFGLISSLRVVGVVIAVGIFLFLIALVGLIGAVKHHQVLLFFYMIILLLVFIVQFSVSCACLALNQEQQGQLLEVGWNNTASARNDIQRNLNCCGFRSVNPNDTCLASCVKSDHSCSPCAPIIGEYAGEVLRFVGGIGLFFSFTEILGVWLTYRYRNQKDPRANPSAFL

SEQ ID NO: 23 - protein, 25 aa, Mus musculus
  Tetraspan Net6 (SAS)-y2H
EILGVWLTYRYRNQKDPRANPSAFL

SEQ ID NO: 24 - protein, 103 aa, Mus musculus
  Mouse ATPase H+ transporting mitochondrial protein-7304906
MAKFIRNFAEKAPSMVAAAVTYSKPRLATFWHYAKVELVPPTPAEIPTAIQSVKKMIQSAKTGSFKHLTVKEAVLNGLVATEVWMWFYIGEIIGKRGIVGYDV

SEQ ID NO: 25 - protein, 103 aa, Homo sapiens
  Human ATPase H+ transporting mitochondrial protein- AAD20961
MAQFVRNLVEKTPALVNAAVTYSKPRLATFWYYAKVELVPPTPAEIPRAIQSLKKIVNSAQTGSFKQLTVKEAVLNGLVATEVLMWFYVGEIIGKRGIIGYDV

SEQ ID NO: 26 - protein, 61 aa, Mus musculus
  Mouse ATPase H+ transporting mitochondrial protein-y2H
PAEIPTAIQSVKKMIQSAKTGSFKHLTVKEAVLNGLVATEVWMWFYIGEIIGKRGIVGYDV

SEQ ID NO: 27 - protein, 69 aa, Mus musculus
  Mouse Cytochrome C Oxidase 8a (Cox8a)- 6680992
MSVLTPLLLRSLTGSARRLMVPRAQVHSKPAREQLGVLDITIGLTSCFVCCLLPAGWVLSHLESYKKRE

SEQ ID NO: 28 - protein, 69 aa, Homo sapiens
  Human Mouse Cytochrome C Oxidase 8a (Cox8a)- XP_006132
MSVLTPLLLRGLTGSARRLPVPRAKIHSLPPEGKLGIMELAVGLTSCFVTFLLPAGWILSHLETYRRPE

SEQ ID NO: 29 - protein, 53 aa, Mus musculus
  Mouse Cytochrome C Oxidase 8a (Cox8a)-y2H
RRLMVPRAQVHSKPAREQLGVLDITIGLTSCFVCCLLPAGWVLSHLESYKKRE

SEQ ID NO: 30 - protein, 316 aa, Mus musculus
  Mouse Aldehyde-ketone reductase-6753037
MASHLELNNGTKMPTLGLGTWKSPPGQVTEAVKVAIDLGYRHIDCSQVYQNEKEVGVALQEKLKEQVVKRQDLFIVSKLWCTFHDKSMVKGAFQKTLSDLQLDYLDLYLIHWPTGFKPGPDYFPLDASGNVIPSDTDFVDTWTAMEQLVDEGLVKTIGVSNFNPLQIERILNKPGLKYKPAVNQIECHPYLTQEKLIEYCHSKGIVVTAYSPLGSPDRPWAKPEDPSLLEDPRIKAIAAKYNKTTAQVLIRFPIQRNLVVIPKSVTPVRIAENLKVFDFEVSSEDMATLLSYNRNWRVCALMSCAKHKDYPFHAEV

SEQ ID NO: 31 - protein, 176 aa, Mus musculus
  Mouse Aldehyde-ketone reductase-y2H
TWTAMEQLVDEGLVKTIGVSNFNPLQIERILNKPGLKYKPAVNQIECHPYLTQEKLIEYCHSKGIVVTAYSPLGSPDRPWAKPEDPSLLEDPRIKAIAAKYNKTTAQVLIRFPIQRNLVVIPKSVTPVRIAENLKVFDFEVSSEDMATLLSYNRNWRVCALMSCAKHKDYPFHAEV

SEQ ID NO: 32 - protein, 215 aa, Mus musculus
  Mouse TRF-alpha (NAC)-U22151
MPGEATETVPATEQELPQPQAETGSGTESDSDESVPELEEQDSTQTATQQAQLAAAAEIDEEPVSKAKQSRSEKKARKAMSKLGLRQVTGVTRVTIRKSKNILFVITKPDVYKSPASDTYIVFGEAKIEDLSQQAQLAAAEKFKVQGEAVSNIQENTQTPTVQEESEEEEVDETGVEVKDIELVMSQANVSRAKAVRALKNNSNDIVNAIMELTM

SEQ ID NO: 33 - protein, 219 aa, Mus musculus
  Mouse TRF-alpha (NAC)-y2H
SPHKMPGEATETVPATEQELPQPQAETGSGTESDSDESVPELEEQDSTQTATQQAQLAAAAEIDEEPVSKAKQSRSEKKARKAMSKLGLRQVTGVTRVTIRKSKNILFVITKPDVYKSPASDTYIVFGEAKIEDLSQQAQLAAAEKFKVQGEAVSNIQENTQTPTVQEESEEEEVDETGVEVKDIELVMSQANVSRAKAVRALKNNSNDIVNAIMELTM

SEQ ID NO: 34 - protein, 204 aa, Mus musculus
  Mouse CGI-98-AF151856
MAKVQVNNVVVLDNPSPFYNPFQFEITFECIEDLSEDLEWKIIYVGSAESEEYDQVLDSVLVGPVPAGRHMFVFQADAPNPGLIPDADAVGVTVVLITCTYRGQEFIRVGYYVNNEYTETELRENPPVKPDFSKLQRNILASNPRVTRFHINWEDNTEKLEDAESSNPNLQSLLSTDALPSASKGWSTSENSLNVMLESHMDCM

SEQ ID NO: 35 - protein, 219 aa, Mus musculus; identical to SEQ ID NO: 33
  Mouse CGI-98-y2H

SEQ ID NO: 36 - DNA, 412 bp, Mus musculus
  misc_feature 26...411: n=A, C, G and T
ggcacgaggcctcgtgccgaattcgnnacgaggnnnngagagaactantntcgangcatgcatntagaggcccgcatcatgtaattatnttatgtcactgcttacattcacagcattgnggnctttntggataantatcctcccccaagtttgggatgagactactanatgnngactgggntntgacncttgnttaagcactttattaagggcattgacttgngtggtgnngtgggtaacccccattcttccacagcctgcaagggaaggncttccanaaaggctgtggactggggnttgggngagactagccannagtgagccctgcacancaaggtaatgggggttgngcanagacagncctagatctggntttgtaatgatttatncnnnaaataaanannccnnnnnc

SEQ ID NO: 37 - DNA, 601 bp, Mus musculus
  misc_feature 10...577: n=A, C, G and T
ggcacgaggnanagaactaacacttagangcatgcatctagagngccgnatcntgaanntattcctttgctcacaaggtgttgctttgtcctcgtgatgagtttataagccttcttccctccctgctccttaacaaaaacaatgtgatggatcggtgatgttcacagttctccctgtgcggatgcacttgtgctcatagggtttggtaagatgatctcaagtaactacaccagctttggtgtagcagtgaactccgggtggtgctgggatctgatccaacgaaaaacacttgccattctcaccgaagagaactggctttgggactctgttaactttggggaaggaaacattgtatgtttggctcagtgtgcttggctggttaatctttggtaatttcacctgaatgagatttgatgaattaatgaatattgaatttaaaactaattatgagtgaaaaataaaagggncgngcttaaaaaggtatgtcacgctnacnttcacgcacccccgtggggcacgttccatgtgttcncnggccnggacccaagcgtnacaggcattacaactntgcccgagnctgagatggctctaaaaaaaaaaa

SEQ ID NO: 38 - DNA, 256 bp, Mus musculus
  misc_feature 113: n=A, C, G and T
ggcacgagggtagcaattgaaatgagaaatcagttcatcatttcaagtactacattgcctgcattacaaattctctgatcacagaattgtacataaccctgagcctatgtacntgccatttgattgtcaggttccatttgtgtcaaaacctaaagatagtgatgaccaagtgaaacagagtggtgtattgctttttcatatgaatattttcacataaaagcatttgtgaaagcaaaaaaaaaaaaaaaaaaaaaaa

SEQ ID NO: 39 - DNA, 545 bp, Mus musculus
  misc_feature 204...541: n=A, C, G and T
ggcacgaggcagtatctcccagtagcacttcttctggatttgaattcaaatgtcattcttaacatttctgttgatgcttctttgaaaatcttcgcttgattttggctgtaaatcacctaccctgttaaatgcttgtttaatcccaaggatgtaagttgaggtgaggtgtcttctctgtctgccccattgcactcaactgaatanaaatatttggntttagagcatctcaaaagtgggtagaatgtcctttgggattttgttgctccgtttaaaccaggtgtatngcgacatgaacatgactttcctaaagtggaaggatctgtccccaccttcacccagtccttangacaagtagaaaggggcancctggcacttggcataacctatgccaccanggcnagataactgtgaaaaagnggtatcctctgatgtcccncgtattagnctcnnnttacaaancacttaataaaactancctactgtnnaaccancnccaaaaccnacaaancccccgccccttttttttnnancggncccccnntaga

SEQ ID NO: 40 - DNA, 477 bp, Mus musculus
  Mouse TSAP6int.EST.Cl.23(200)same as Cl.40-AA915693
ccagtagcacttcttctggatttgaattcaaatgtcattcttaacatttctgttgatgcttctttgaaaatcttcgcttgattttggctgtaaatcacctaccctgttaaatgctgtttaatcccaaggatgtaagttgaggtgaggtgtcttctctgtctgccccattgcactcaactgaatagaaatatttgggtttagagcatctcaaaagtgggtagaatgtcctttgggattttgttgctccgtttaaaccaggtgtattgtgacatgaacatgactttcctaaagtggaaggatctgtccccaccttcacccagtccttaggacaagtagaaaggggcattctggcacttggcataacctatgccacgatggtgagataatgtgaaaaagtggtatcctctgatgtcagcgtattagtctcagtttacaaagtacttaataaaactaactactgtaaaaccaaaaaaaaaa

SEQ ID NO: 41 - DNA, 733 bp, Mus musculus
  Mouse TSAP6int.EST.Cl.62(342)-AA537195
gaattcggcacgagggtgtgcttggcctctgttagactcctgtgtggttccacaatgcctaagaaagccggtgcgacaagcaagggtaaaaaccaaaccaaagaaccagagacagcacctcctgctgcagggcctgtagcaactgaccctaaaggttttgtcaccatagccattcatgccaaacctggctccagacagaacgctgtgacagatctgagcactgaggccgttggtgtggccattgcagcacctccgtcccagggagaggcaaatgcggagctgtgtcggtacctctccaaggtcttggacctcaggaagagtgacgtggttctggataagggtggtaaatctcgggaaaaggtggtgaagctcttggcctctacaactccagaggaggtcttggagaagctgaaaacagaagctgagaagaaataaaggcgagaaatggaaacagctgctgtctgcgacggagcatctgagctgcaggcactgggagaggagctgaaggtcgccgcgtggaggcggacaggacaccagctccctggactccgagtgaagctttagctttatagctccacaatgttttgtggagttctggggtctcctaattcagtttacaactggaaaaccttttgtgctgcttctattctaagtttttggtttttaaatgcaaaagtgttctctttatatcttttaaagattctctggacatttcgtggaaaagtaaataatc

SEQ ID NO: 42 - DNA, 611 bp, Mus musculus
  misc_feature 16...575: n=A, C, G and T
gaattcggcacgaggnntanctctaaaanctgatntnngnggcactattanaagntnnactataaggagnttctgnttgccntgtacctagcacttccccagatgngcanataacggagtagncaggtggaatgcactaatgcacccagcattcatgtggggtangaggagcgggnaggtcaaagtggnccttacaaatagcaggctctangccagggtggnctatagatgactctgnntctaacaaacacacaaataaaacattgtgtgaatgatggtattttagtacatggttctctacccagcccctctgtccctcangcattctanactctgtgctgacaggactaaattgctcctacaaaaataatagtactaangtataanccaattctatatttgtgatagaanaaaaaatgaatattctttaacaggaagtatgttcctagaatgggatatcatatggtaaattacagtgataacaatagatctggttgacatgacgttatatttttgtgaagtatttcanaagaatatatattttctcagttttataaaaatgaaaaataaaaganattggttgtttataaaaaaaaaaaaaaanaaaaaaa

SEQ ID NO: 43 - DNA, 548 bp, Mus musculus
  misc_feature 30...85: n=A, C, G and T
gaattcggcacgagggagagagagaactantctcgaggcatgcatctagaggnccgcatcatgtaattagntatgtcacgcttcnttcacgcagtcatatttcataagctcttttggcctgggtacagcagaaaactagcagttttcctacttggcgcaagtgacttgtgcctccttgtctgtcctttgtcagcacaggtgcaggcaaacccttccagctgggttaattgccgtttatggtttactcacttagggaaagggcttaggtgttaggctgtgggctgcttctccctaaccatccattgtgcagacttctcatctaaaaaggttggtggcttttgcttgggatcagtgctctgctaacgcccttgctggtctctccacacattcctgtcattgagacttgaattgtaggtgtgatgttatgcacaggatgctcagagctatgttactactattcttagtttgtaaattgtccttttgataccatcttgttttcttttgtaggtataaataaatacttgacaataaaaaaaaaaaaaaaaaaa

SEQ ID NO: 44 - protein, 338 aa, Mus musculus [sequence omitted]

SEQ ID NO: 45 - protein, 75 aa, Mus musculus
  Mouse TSAP6int. HUKIAA0095-y2H
LSEVLLATMNILFTQFKRLKGTSPSSSSRPQRVIEDRDSQLRSQARTLITFAGMIPYRTSGDTNARLVQMEVLMN

SEQ ID NO: 46 - DNA, 147 bp, Mus musculus
  Mouse Sca-1 Non-coding region NM_008529
gttcaggaggctgcccatggaggactgccacccctccagatgaaggctcccactacccgatgcagttgagtcccatcctgccctctctgcccacactggcttcctgctgctattctagtgcctcaaataaaccgttcacacccttaa

SEQ ID NO: 47 - protein, 50 aa, Mus musculus
  misc_feature 49: Xaa=any amino acid
VQEAAHGGLPPLQMKAPTTRCSXVPSCPLCPHWLPAAILVPQINRSHPXK

SEQ ID NO: 48 - DNA, 133 bp, Mus musculus
  Mouse Nebulin related anchoring protein U76618
ggcacgaggcttttggatatatttttaattaaaaaaatcacatgagaaatgtaatctgtatagtgatactttaataagcacatactcaataaacaactgcaatgatgtcgacagcaaaaaaaaaaaaaaaaaa

SEQ ID NO: 49 - DNA, 2969 bp, Mus musculus
  Murine sequence TSAP6
cctcggaaacttaggtgaatccacttctagtccagacctttgaggtgggaagacacacctttaatctgggccacaccttctgctgggaggtataaggacgcagacctgctctttgcctgcttgtcctcaccttgctaacacatccactcctcactggcactggggctacttctttgggattccagcatatactgaagaccagctgaaatatctagcctcatagactgaacaactactggatttttggactttctgttcacagtcaggtatactgcagccttgagctccagccacccagcttcagaagaaatggctgcagaggcccacaggcagcagggctcttgccccaccatcccctcagagggctgtggaaagtcaccagagaagaaaggcagtgcggccgactctagacctggcactgctatgtcgggggagatggacaagccgctgatcagccgccgcctagtggacagtgatggcagtctggctgaggtccccaaggaggcccccaaagtgggcatcctgggcagtggggattttgcccgttccctggccacacgcctggtgggctctggcttcagtgtggtggtggggagccgtaaccccaaacgcacggctggcctcttcccctccttagctcaagtgactttccaggaggaagccgtgagctctccagaggtcatctttgtggccgtgttccgggagcactactcctcactgtgcagtcttgctgaccagttggctggcaagatcctcgtggatgtaagcaaccccacggagaaggagcatcttcagcaccgccagtctaatgctgagtacctggcctcactctttcctgcgtgcactgtggtgaaggccttcaacgtcatctctgcatgggccctacaggctggcccaagggatgggaacaggcaggtgctcatctgcagtgatcagccagaagccaagcgcaccatctcagagatggcacgcgccatgggtttcacacccctggacatgggatccctggcctcagcgagggaggtagaagccatacccctgcgcctccttccatcctggaaggtgcccaccctcctggcactggggctctttgtgtgcttctacacctacaacttcatccgggacgttctacagccatacattcggaaagatgagaacaagttctacaagatgcccttgtctgtggtcaacaccacactaccctgtgtggcttatgtgctgctgtccctagtgtacctgcccggtgtgctggcagctgcgcttcagctgcggagggggaccaagtaccagcgcttcccagactggctggaccactggctgcagcatcgcaagcagatcgggctgctcagcttcttcttcgcgatgctgcacgctctctacagcttctgcctgccgctgcgccgctcccaccgctacgacctggtcaatctggctgtgaagcaggtcctggccaacaagagccgcctctgggttgaggaagaagtctggaggatggagatatacctgtccctgggtgtgctggccctgggcatgttgtcgctgctggctgtcacctcgctcccgtccattgccaactccctcaactggaaggagttcagcttcgtgcagtccacgctgggcttcgtggccctgatactcagcacaatgcacacactcacctacggctggacccgtgcctttgaggaaaaccactacaagttctacctgccgcccacattcacactcacgctgctcctgccctgtgtgatcatcctggccaagggcctcttcctcctgccctgcctcagccgcagactcaccaagatccgcaggggctgggagaaagatggggctgtcaagttcatgctgcccggcgaccacacacagggggagaaaacaagccacgtgtgaggccctggaagcggagatggcttgtgggggccctgagctgggttcgggtctcttttctggatgctgcacagcgaggtgatgatatatgcgtgggtggctgagatcctaattcctggggtgcaggtgtaaactgacatactcagaatgacaccccatacatgtgatatgtactcacatatatttcacatataataagatttgctattattcttacttagctaaaaaaaaaaagtgggtccctatatttcagcgtaagcatttcaaagcaaatgccacacattgaacagcagatcccacccttgtggtatctacagaggcagacagacactctggtataggagaaactgtctttcgttggattctctcctttaatctctatgctccttattagctgaatcctaaagttggtgcaaagctggggcaagaaatgcctctggtgccacctacccccatcccagggctaagaaagaagcctcgagtgaacagggaaccaggtctggactctgctgcttccctgggcgtgcgtggggaggctcagcaagaccccctgggatctatgcaggagctttttcaggtccgtcctttcttcagggaagggtctgaagctgccccatctgatcctagctgagctgagaagattcttccccaccccctgaaagtccagagtcaccaccggagcctgcaaattgatccttctgcgaaggtgtgaagtcaccgcctctccagagccattaatgaacctggtcttcgggaggaggataattgtttcctctccattaagttgctggtgacccccctttaaatcactgtgccttctcgccttttccatcattaatttggacatctccgtggagtggacacttgtctgggcagttcggggggggggggagcattagagattgcagagaataaccatcgaatcctcttcttggggcaaccctccccttggatgtgccccaggcctgccttcattaaattggtccctgaggagaaaaaaaaaaaaaaaaaaaaaaaaaaaaaaaaaaaaaaaaaaaaatttaaaaaaaaaaaaaaaaaaaaaaaaaaaaaaaaaaaaaaaa

SEQ ID NO: 50 - protein, 526 aa, Mus musculus
  Murine TSAP6 protein
MAAEAHRQQGSCPTIPSEGCGKSPEKKGSAADSRPGTAMSGEMDKPLISRRLVDSDGSLAEVPKEAPKVGILGSGDFARSLATRLVGSGFSVVVGSRNPKRTAGLFPSLAQVTFQEEAVSSPEVIFVAVFREHYSSLCSLADQLAGKILVDVSNPTEKEHLQHRQSNAEYLASLFPACTVVKAFNVISAWALQAGPRDGNRQVLICSDQPEAKRTISEMARAMGFTPLDMGSLASAREVEAIPLRLLPSWKVPTLLALGLFVCFYTYNFIRDVLQPYIRKDENKFYKMPLSVVNTTLPCVAYVLLSLVYLPGVLAAALQLRRGTKYQRFPDWLDHWLQHRKQIGLLSFFFAMLHALYSFCLPLRRSHRYDLVNLAVKQVLANKSRLWVEEEVWRMEIYLSLGVLALGMLSLLAVTSLPSIANSLNWKEFSFVQSTLGFVALILSTMHTLTYGWTRAFEENHYKFYLPPTFTLTLLLPCVIILAKGLFLLPCLSRRLTKIRRGWEKDGAVKFMLPGDHTQGEKTSHV

SEQ ID NO: 51 - DNA, 1720 bp, Homo sapiens
  Human sequence TSAP6
acccagccaccaaaatgccagaagagatggacaagccactgatcagcctccacctggtggacagcgatagtagccttgccaaggtccccgatgaggcccccaaagtgggcatcctgggtagcggggactttgcccgctccctggccacacgcctggtgggctctggcttcaaagtggtggtggggagccgcaaccccaaacgcacagccaggctgtttccctcagcggcccaagtgactttccaagaggaggcagtgagctccccggaggtcatctttgtggctgtgttccgggagcactactcttcactgtgcagtctcagtgaccagctggcgggcaagatcctggtggatgtgagcaaccctacagagcaagagcaccttcagcatcgtgagtccaatgctgagtacctggcctccctcttccccacttgcacagtggtcaaggccttcaatgtcatctctgcctggaccctgcaggctggcccaagggatggtaacaggcaggtgcccatctgcggtgaccagccagaagccaagcgtgctgtctcggagatggcgctcgccatgggcttcatgcccgtggacatgggatccctggcgtcagcctgggaggtggaggccatgcccctgcgcctcctcccggcctggaaggtgcccaccctgctggccctggggctcttcgtctgcttctatgcctacaacttcgtccgggacgttctgcagccctatgtgcaggaaagccagaacaagttcttcaagctgcccgtgtccgtggtcaacaccacactgccgtgcgtggcctacgtgctgctgtcactcgtgtacttgcccggcgtgctggcggctgccctgcagctgcggcgcggcaccaagtaccagcgcttccccgactggctggaccactggctacagcaccgcaagcagatcgggctgctcagcttcttctgcgccgccctgcacgccctctacagcttctgcttgccgctgcgccgcgcccaccgctacgacctggtcaacctggcagtcaagcaggtcttggccaacaagagccacctctgggtggaggaggaggtctggcggatggagatctacctctccctgggagtgctggccctcggcacgttgtccctgctggccgtgacctcactgccgtccattgcaaactcgctcaactggagggagttcagcttcgttcagtcctcactgggctttgtggccctcgtgctgagcacactgcacacgctcacctacggctggacccgcgccttcgaggagagccgctacaagttctacctgcctcccaccttcacgctcacgctgctggtgccctgcgtcgtcatcctggccaaagccctgtttctcctgccctgcatcagccgcagactcgccaggatccggagaggctgggagagggagagcaccatcaagttcacgctgcccacagaccacgccctggccgagaagacgagccacgtatgaggtgcctgccctgggctctggaccccgggcacacgagggacggtgccctgagcccgttaggttttcttttcttggtggtgcaaagtggtataactgtgtgcaaataggaggtttgaggtccaaattcctgggactcaaatgtatgcagtactattcagaatgatatacacacatatgtgtatatgtatttacatatattccacatatataacaggatttgcaattatacatagctagct

SEQ ID NO: 52 - protein, 488 aa, Homo sapiens
  Human TSAP6 protein
MPEEMDKPLISLHLVDSDSSLAKVPDEAPKVGILGSGDFARSLATRLVGSGFKVVVGSRNPKRTARLFPSAAQVTFQEEAVSSPEVIFVAVFREHYSSLCSLSDQLAGKILVDVSNPTEQEHLQHRESNAEYLASLFPTCTVVKAFNVISAWTLQAGPRDGNRQVPICGDQPEAKRAVSEMALAMGFMPVDMGSLASAWEVEAMPLRLLPAWKVPTLLALGLFVCFYAYNFVRDVLQPYVQESQNKFFKLPVSVVNTTLPCVAYVLLSLVYLPGVLAAALQLRRGTKYQRFPDWLDHWLQHRKQIGLLSFFCAALHALYSFCLPLRRAHRYDLVNLAVKQVLANKSHLWVEEEVWRMEIYLSLGVLALGTLSLLAVTSLPSIANSLNWREFSFVQSSLGFVALVLSTLHTLTYGWTRAFEESRYKFYLPPTFTLTLLVPCVVILAKALFLLPCISRRLARIRRGWERESTIKFTLPTDHALAEKTSHV

(19) United States
(12) Patent Application Publication
(10) Pub. No.: US 2004/0175772 A1
(43) Pub. Date: Sep. 9, 2004

(54) Screening method based on tsap 6 binding partners

(76) Inventors: Robert Amson, Paris (FR); Adam Teleman, Paris (FR); Brent Passer, Paris (FR)

Correspondence Address: JONES DAY, 51 Louisiana Aveue, N.W, WASHINGTON, DC, 20001-2113, US

(21) Appl. No.: 10/451,861

(22) PCT Filed: Dec. 24, 2001
(86) PCT No.: PCT/FR01/04188

(30) Foreign Application Priority Data
Dec. 26, 2000 (FR) 00/17027
Sep. 18, 2001 (FR) PCT FR01/02896

(51) Int. Cl. C12Q 1/68; G01N 33/574; C12N 9/64

(52) U.S. Cl. 435/7.23; 435/226; 435/6

(57) ABSTRACT

The invention concerns methods for detecting, identifying and/or screening compounds in particular for use in treating cancers and certain neurodegenerative diseases related to dysfunction in tumour suppression regulation and/or apoptosis, in the biological chain of p53, said methods being based on interaction between TSAP6 and its binding partners identified in the present invention.

The present invention relates to methods for detecting, identifying and/or screening compounds which may in particular be used for treating cancers or some neurodegenerative diseases related to dysfunction in the regulation of tumor reversion/suppression and/or apoptosis, in the p53 biological pathway.

Apoptosis, or cell death, is a complex phenomenon which is regulated by many proteins, including p53. This protein interacts with many other proteins, and its expression, which induces the phenomena of cell death and of tumor reversion, can be correlated with the induction or the suppression of expression of other cellular genes.

The inventors of the present invention have thus demonstrated genes which are induced and activated during the cascade leading to tumor reversion and/or apoptosis (TSAP for “Tumor Suppressor Activated Pathway”), or genes which are suppressed (TSIP for “Tumor Suppressor Inhibited Pathway”). These genes have in particular been the subject of patent application WO 97/22695 or WO 00/08147.

It is important to be able to understand precisely the mechanisms of the p53 cascade, in order to be able to generate new compounds having antitumor activity (which can in particular induce apoptosis or tumor suppression), or which can be used for the treatment of neurodegenerative diseases. Specifically, the inventors of the present application have demonstrated that presenilin 1 (PS1), the role of which in Alzheimer's disease had been suggested, is identical to the TSIP 2 protein described in application WO 97/22695. Thus, it is legitimate to search for medicinal products which can interfere in apoptosis, in order to reduce this phenomenon, and which might be used in neurodegenerative diseases.

The present invention relates to methods for screening and identifying products which can interfere in the p53 cascade and thus induce tumor reversion and/or apoptosis or, conversely, decrease the phenomena of apoptosis.

The present invention is based on the interactions of the TSAP6 protein with other proteins, as demonstrated by the inventors of the present application. The TSAP6 protein, described in patent application WO 97/22695, and in GenBank under the number U50961, is a protein with six transmembrane domains, which suggests that it is located in a cell membrane. Thus, it is reasonable to think that the TSAP6 protein can act as a receptor in the metabolism for regulating p53-associated apoptosis, and that determining its binding partners may prove to be important with regard to the overall understanding of the regulation of apoptosis/tumor reversion in cells.

The nucleotide sequence of murine TSAP6 is represented by SEQ ID No 49 and the open reading frame of the protein corresponds to SEQ ID No 50.

The nucleotide sequence of human TSAP6 is represented by SEQ ID No 51 and the open reading frame of the protein corresponds to SEQ ID No 52.

Thus, in a first embodiment, the present invention relates to methods of screening and/or selecting or identifying a compound which interferes with, reduces or inhibits the binding of TSAP6 to one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, having the steps of:

a) bringing said compound into contact with a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;

b) identifying the decrease in and/or the inhibition of the binding between TSAP6 and said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48.

In order to determine compounds which make it possible to increase tumor reversion and/or cell death (apoptosis), it is also possible to define a method according to the invention, in particular a method for screening, selecting or identifying compounds whose function is an increase in tumor reversion and/or cell death (apoptosis), having the steps of:

a) bringing said compound into contact with a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;

b) identifying the compounds which induce the decrease in and/or the inhibition of the binding between TSAP6 and said protein defined in a);

c) bringing the compounds selected in step b) into contact in a system for measuring the phenomena of apoptosis and/or of tumor reversion;

d) identifying the increase in tumor reversion and/or in cell death (apoptosis) in said model by comparison with a control model with which said compound has not been brought into contact.

Such a method is therefore also a subject of the present invention.

The subject of the present invention is also a method for screening, selecting or identifying compounds whose function is a decrease in and/or the inhibition of tumor reversion and/or cell death (apoptosis), having the steps of:

a) bringing said compound into contact with a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;

b) identifying the compounds which induce the decrease in and/or the inhibition of the binding between TSAP6 and said protein defined in a);

c) bringing the compounds selected in step b) into contact in a system for measuring the phenomena of apoptosis and/or of tumor reversion;

d) identifying the decrease in and/or the inhibition of tumor reversion and/or cell death (apoptosis) in said model by comparison with a control model with which said compound has not been brought into contact.

The present invention therefore uses the fact that the TSAP6 proteins and the proteins chosen from SEQ ID No 1 to SEQ ID No 35 or SEQ ID No 44 or 46, or encoded by a nucleic acid chosen from SEQ ID No 36 to SEQ ID No 43 or SEQ ID No 45 or 47, can bind to one another. It is therefore advantageous to identify the domains of each protein which are effectively in contact with the other protein. As a result, this should make it possible to be able to use the peptides thus identified as decoys or agonists for the complete proteins. This may thus make it possible to define compounds which will interfere in the binding between TSAP6 and one of the proteins chosen from-SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, and which may either induce tumor suppression and/or apoptosis or, conversely, decrease these phenomena.

A subject of the present invention is therefore in particular a method for identifying a region of TSAP6 which binds with one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, comprising the steps of:

a) bringing peptides derived from the TSAP6 protein into contact in a system for evaluating the binding of the TSAP6 protein with said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;

b) identifying the peptides which induce the decrease in the binding between the TSAP6 protein and said protein defined in a) by comparison with the binding observed between TSAP6 and said protein defined in a), when said peptides are absent from the system.

The term “region of TSAP6 ” is in particular intended to mean peptides with a primary sequence derived from the primary sequence of the TSAP6 protein.

A subject of the present invention is also of course the methods for identifying the regions of the proteins chosen from SEQ ID No 1 to SEQ ID No 35 or SEQ ID No 44 or 46, or encoded by a nucleic acid chosen from SEQ ID No 36 to SEQ ID No 43 or SEQ ID No 45 or 47, which binds with TSAP6.

The system envisioned can be implemented in vitro or in vivo.

Thus, the present invention makes it possible to identify regions of TSAP6 which are involved in the binding with one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, using a method comprising the steps of:

a) bringing peptides derived from the TSAP6 protein into contact in a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;

b) identifying the peptides which lead to the decrease in the binding between TSAP6 and said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, in said system.

It is obvious that the present invention also makes it possible to determine the regions of one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, which are involved in the binding with TSAP6, according to methods similar to the methods described above, and that these regions can in particular be used as decoys when the intention is to decrease tumor reversion and/or apoptosis.

The present invention therefore makes it possible to identify products which make it possible to interact with the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, and which may therefore be of value in the regulation of apoptosis and/or tumor reversion. However, it is possible that these products, in order to be able to be used for a therapeutic treatment, in particular in cancer or neurodegenerative diseases, need to be optimized in order to have greater activity and/or less toxicity.

In fact, a medicinal product is often developed according to the following principle:

screening of compounds having a desired activity using a suitable method,

selection of the compounds corresponding to the “specifications”,

determination of the structure (in particular the (optionally tertiary) sequence if they are peptides, formula and backbone if they are chemical compounds) of the compounds selected,

optimization of the compounds selected, by modification of the structure (for example by changing the stereochemical conformation (for example changing from L to D for the amino acids in a peptide), addition of substituents to the peptide or chemical backbones, in particular by grafting residues onto the backbone, modification of the peptides (see in particular Gante (“Peptidomimetics”, in Angewandte Chemie-International Edition Engl. 1994, 33. 1699-1720)),

passage and screening of the compounds thus obtained on suitable models which are often models closer to the pathology condition studied. At this stage, animal models, in general in rodents (mice, rats, etc.) or in dogs, or even primates, are in particular often used.

The animal models which can be used are for example, for cancer, models based on immunodepressed mice (for example scid/scid), into which tumor cells are injected (in particular subcutaneously), which cells will lead to the development of tumors. The effectiveness of the potentially antitumor compounds is studied, for example by measuring the size of the tumors formed.

For studying neurodegenerative diseases, the model described by Amson et al. (2000, Proc. Natl. Acad. Sci. USA, 97, 5346-50), which consists of p53-deficient mice, or the model described in Chen et al., Janus et al., and Morgan et al. (2000, Nature, 408 pp. 975-985) can be used.

Thus, a object of the present invention is in particular to make it possible to identify compounds which might be used for treating cancer in that they have an activity of increasing tumor reversion and/or apoptosis. One of the subjects of the present invention is therefore a method comprising the steps of:

a) implementing a method according to the invention which makes it possible to identify compounds having a certain activity of increasing tumor reversion and/or apoptosis, and/or of inhibiting the binding between TSAP 6 and a protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48,

b) modifying the product selected in step a),

c) testing the product modified in step b) in in vitro and/or in vivo methods on relevant models of tumor reversion and/or apoptosis,

d) identifying the product which makes it possible to obtain an activity of tumor reversion and/or apoptosis greater than the activity obtained for the product selected in step a).

Step d) can be replaced with a step d′), which would be:

d′) identifying the product which makes it possible to obtain the desired biological effect with less toxicity in an animal model (when one of the models used in step c) is in vivo).

When the tests are carried out on models of apoptosis or tumor reversion in vitro, the K256/KS model described by Tellerman et al. (1993, Proc. Natl. Acad. Sci. USA, 90, 8702-6) can for example be used. The M1-LTR cells described by Amson et al. (1996, Proc. Natl. Acad. SCI; USA, 93, 3953-7), or the U937/US3-US4 cells described by Nemani et al. (1996, Proc. Natl. Acad. Sci. USA, 93, 9039-42), can also be used.

The in vivo trials can be carried out by injecting these cells into animals (in particular immunodepressed mice), and studying the effects of the various compounds tested.

Those skilled in the art will be able to define the conditions and the thresholds necessary for identifying a product which can be used as a medicinal product, according to the regulatory requirements (in particular for toxicology), with respect to the benefit provided by the product thus identified.

Similarly, the invention also relates to the methods for optimizing the products which suppress tumor reversion and/or apoptosis, identified using the methods described above, and making it possible to identify products which can be used as medicinal products.

Thus, the invention also relates to a method for identifying a product having an activity of decreasing and/or inhibiting tumor reversion and/or apoptosis, characterized in that it comprises the steps of:

a) implementing a method according to the invention which makes it possible to identify compounds having a certain activity of decreasing tumor reversion and/or apoptosis, and/or of inhibiting the binding between TSAP6 and a protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48,

b) modifying the product selected in step a), in particular by grafting residues onto the chemical backbone,

c) testing the product modified in step b) in in vitro and/or in vivo methods, on relevant models of tumor reversion and/or apoptosis,

d) identifying the product which makes it possible to obtain an activity of tumor reversion and/or apoptosis which is decreased compared to the activity obtained for the product selected in step a).

Step d) can also be replaced with a step d′), which would be:

d′) identifying the product which makes it possible to obtain the desired biological effect with less toxicity in an animal model (when one of the models used in step c) is in vivo).

This in fact again involves being able to obtain the product which exhibits the best (biological activity and clinical effect)/(potential risks for use) ratio.

The parameters to be brought into play for obtaining these results are all known and within the scope of those skilled in the art who wish to develop new medicinal products, and can be found, for example, in the directives of the organizations such as the Agence du Médicament [French Drug Agency], the European Commission or the Federal Drug Agency.

The implementation of the methods according to the present invention requires models which make it possible to determine the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48.

When the methods according to the invention are implemented on in vitro models, there are several ways in which the binding between one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, and TSAP6 can be studied.

A protocol which can be used may be as follows:

expression and purification of the TSAP6 proteins and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, for example in prokaryotic cells (E. coli, B. subtilis, etc.) or eukaryotic cells (yeast such as Saccharomyces, Kluyveromyces, etc.), mammalian cells (HeLa, Cos, Hep-2, etc.) or insect cells (using a Baculovirus system). It may be advantageous for the proteins to have a tag at their N- or C-terminal end, in order to facilitate purification. A histidine or GST tag is in particular chosen. These methods are well known to those skilled in the art, who can find the suitable plasmids in the catalogues of companies such as Stratagéne;

binding of the proteins to suitable beads. When a GST tag is used, the proteins expressed are bound to sepharose beads exhibiting glutathione;

preparation of proteins by in vitro translation. This can easily be carried out using commercial vectors (for example available from Promega), which make it possible to clone the cDNAs under the control of well-known promoters (T7 or T3), and to use suitable RNA polymerases to produce the RNAs, and then to carry out expression of the proteins in vitro, using available kits and following the manufacturer's indications;

Coprecipitation of the proteins, by adding the proteins obtained by in vitro translation to the sepharose-glutathione beads to which the proteins from fusion with GST are attached. After a sufficient amount of contact time, the beads are washed and analysis by SDS-PAGE gel electrophoresis and autoradiography is carried out. The appearance of the bands corresponding to the two proteins clearly shows binding between them.

The use of suitable controls thus makes it possible to define the decrease in and/or the inhibition of the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, by comparing the amounts of proteins released after addition of the compound tested during the coprecipitation step, with the amounts of proteins released in the controls.

The binding of the proteins can also be studied using the FRET (Fluorescence Resonance Energy Transfer) system, which consists in labeling each of the proteins with a suitable residue, the binding of the two proteins inducing a reaction between each of the two residues and emission of a readily detectable fluorescence.

A subject of the present invention is also the compounds which can be obtained using a method according to the invention, in particular the compounds having an activity of increasing tumor reversion and/or apoptosis, those having an activity of inhibiting the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, and also those having an activity of decreasing and/or inhibiting tumor reversion and/or apoptosis.

The present invention also relates to the peptide sequences corresponding to a region of TSAP6 which interacts with one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, which can in particular be identified using a method according to the invention.

The invention also relates to the peptide sequences corresponding to a region of one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, which interacts with the TSAP6 protein, which can in particular be identified using a method according to the invention, the method which makes it possible to identify the peptide sequences of TSAP6 which interact with one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, possibly being adapted to determine the peptide sequences of one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, which interacts with TSAP6, in particular by adapting the in vitro protocol developed above.

The invention also relates to the nucleotide sequences encoding the peptide sequences thus identified.

It is clear that the term “peptide sequence” or “nucleic acid sequence” or “nucleotide sequence” (the latter two terms being used indifferently) represents sequences which are isolated, i.e. which are outside their natural state, and can in particular be modified by replacement of their base units with unnatural units, or by modification of the bonds between the base units (for example phosphorothioates (nucleic acid) or Peptide Nucleic Acids).

An object of the present invention is therefore in particular to make it possible to identify compounds which interfere with the binding of TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, some of these compounds being able in particular to induce effects on the p53 cascade. Thus, the compounds according to the invention, the peptide sequences according to the invention or the nucleotide sequences according to the invention, as a medicinal product, are also subjects of the invention.

A compound identified using a method according to the invention may be a compound which has a chemical structure, a lipid, a sugar, a protein, a peptide, a protein-lipid, protein-sugar, peptide-lipid or peptide-sugar hybrid compound, or a protein or a peptide to which chemical branches have been added.

Among the chemical compounds envisioned, they may contain one or more (in particular 2 or 3) rings, which may or may not be aromatic, having from 3 to 8 carbon atoms, and also several residues of any type (in particular lower alkyl, i.e. having between 1 and 6 carbon atoms).

These compounds, nucleic acid sequences and peptide sequences can thus be used, according to the invention, for preparing a medicinal product intended in particular for the treatment of cancer or a neurodegenerative disease, depending on the pro- or anti-apoptosis/tumor reversion effect.

The inventors of the present application have, for the first time, demonstrated the fact that the TSAP6 protein binds to one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48. Thus, the present invention also relates to a complex consisting of a TSAP6 protein and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48.

The present invention also relates to a method for inhibiting the binding of TSAP6 to one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, in a cell, comprising the step of:

a) bringing said cell into contact with a compound identified using a method according to the invention, which inhibits the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48.

The compound thus envisioned can also be a “decoy” peptide derived from the TSAP6 protein or from one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48. The method can be implemented in vitro or in vivo.

The present invention is also directed toward a method for treating a cancer, characterized in that a compound which has been identified according to the present invention and which increases apoptosis and/or tumor reversion is administered to a patient.

A method for treating a neurodegenerative disease, consisting in administering, to a patient, a compound according to the present invention which decreases or inhibits apoptosis, is also a subject of the present invention.

### FIGURE LEGENDS

FIG. 1: TSAP6 colocalizes in the endoplasmic reticulum and golgi

Hela cells transfected with GFP-TSAP6 were analyzed for their subcellular location. Hela cells overexpressing GFP-TSAP6 were colabeled with markers specific for the endoplasmic reticulum (ER), for the golgi, for the mitochondria (mito) and for the nucleus (nuclei). This type of labeling revealed that the subcellular location of TSAP6 is inside the endoplasmic reticulum and the golgi. It should also be noted that TSAP6 also appears to be expressed on the plasma and nuclear membranes.

FIG. 2: TSAP6 antisense prevents p53 induced cell death in TS LTR6 cells

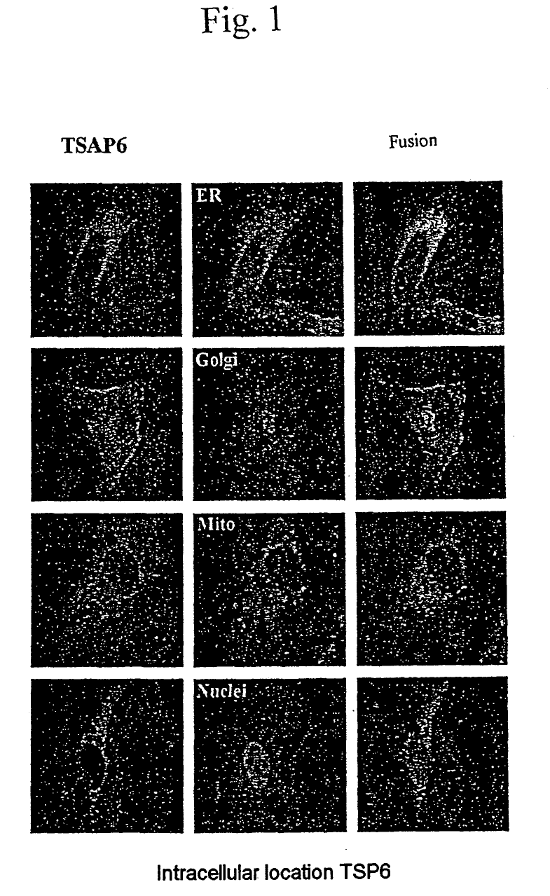

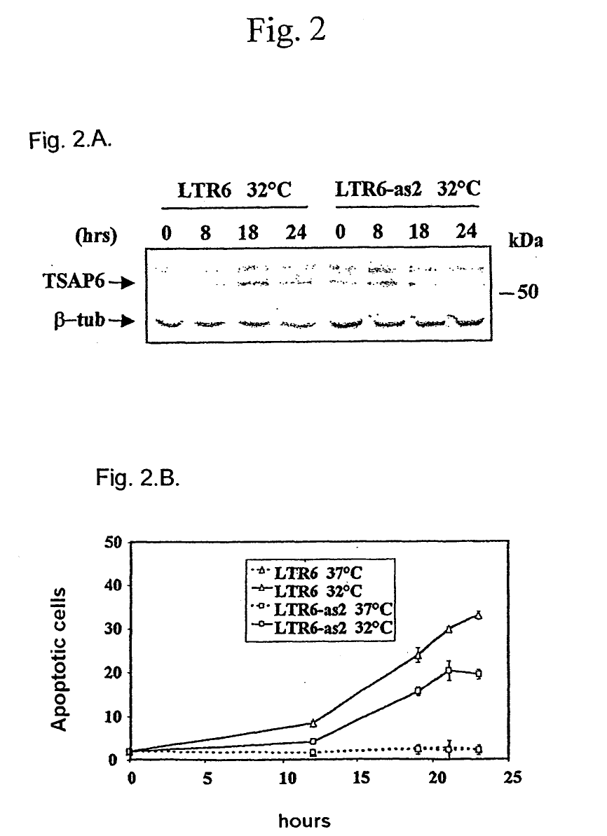

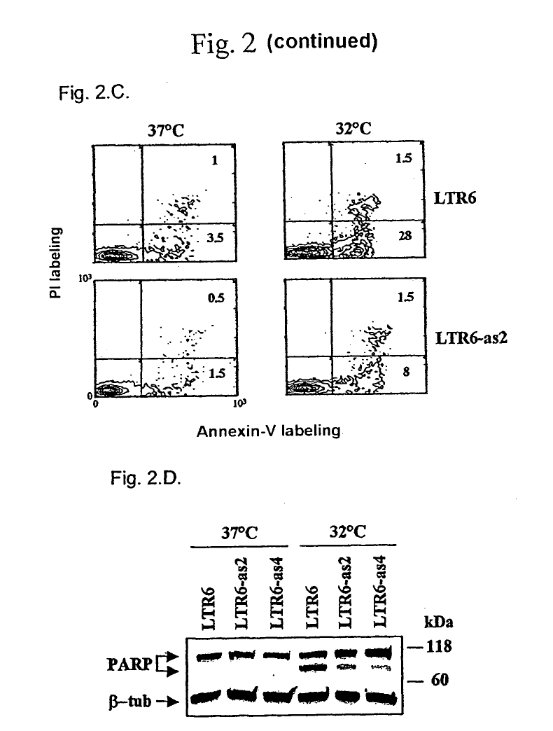

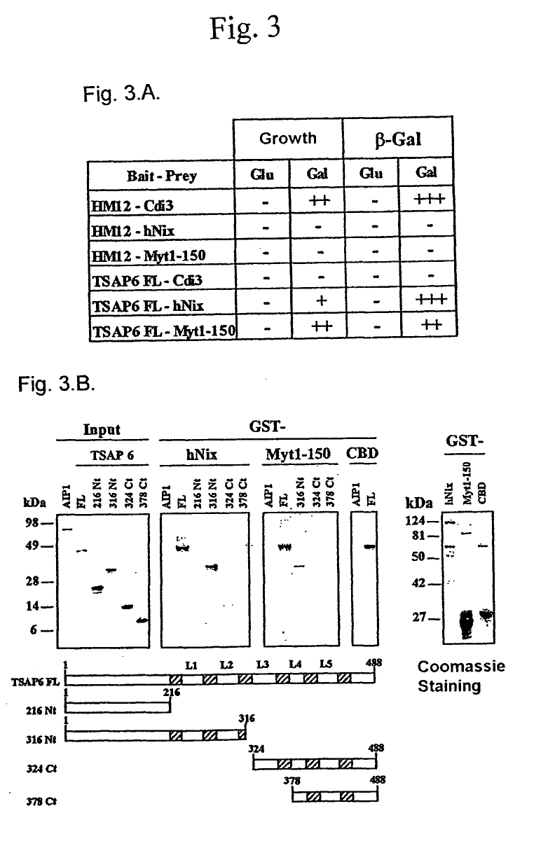

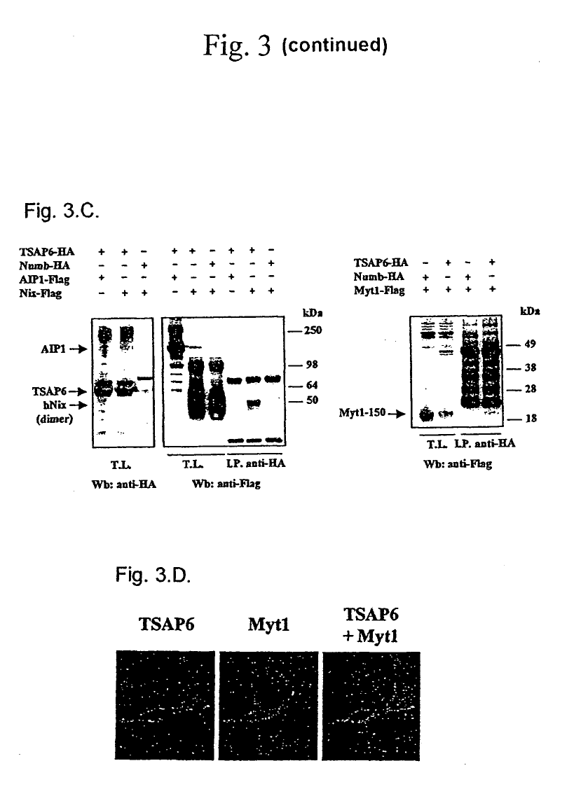

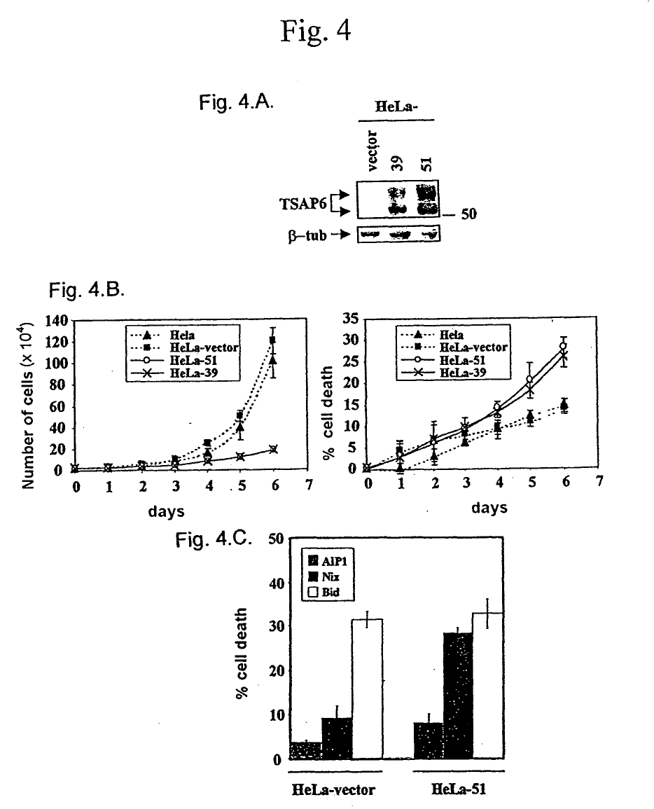

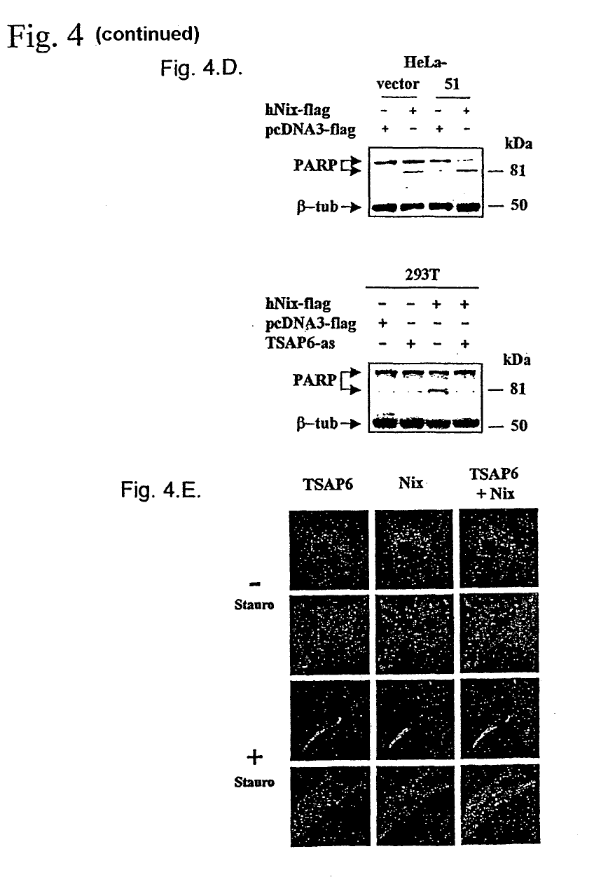

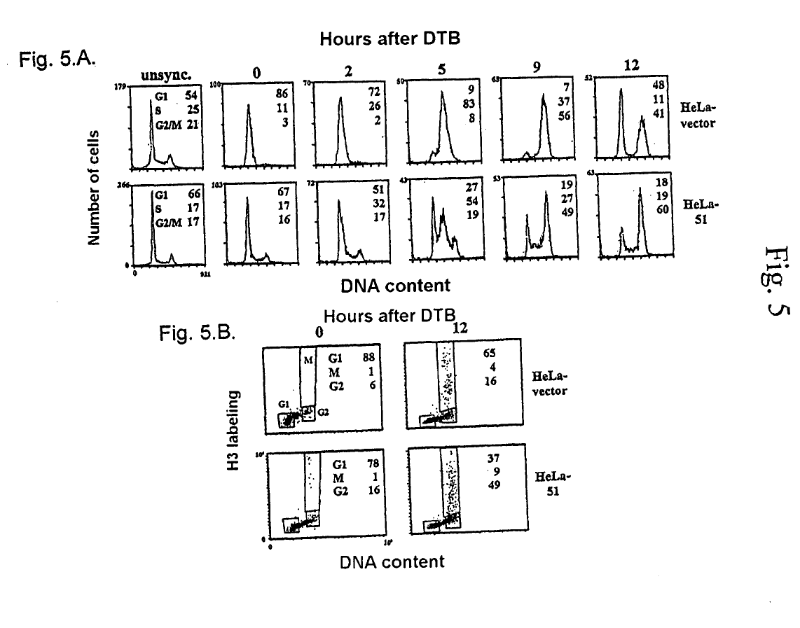

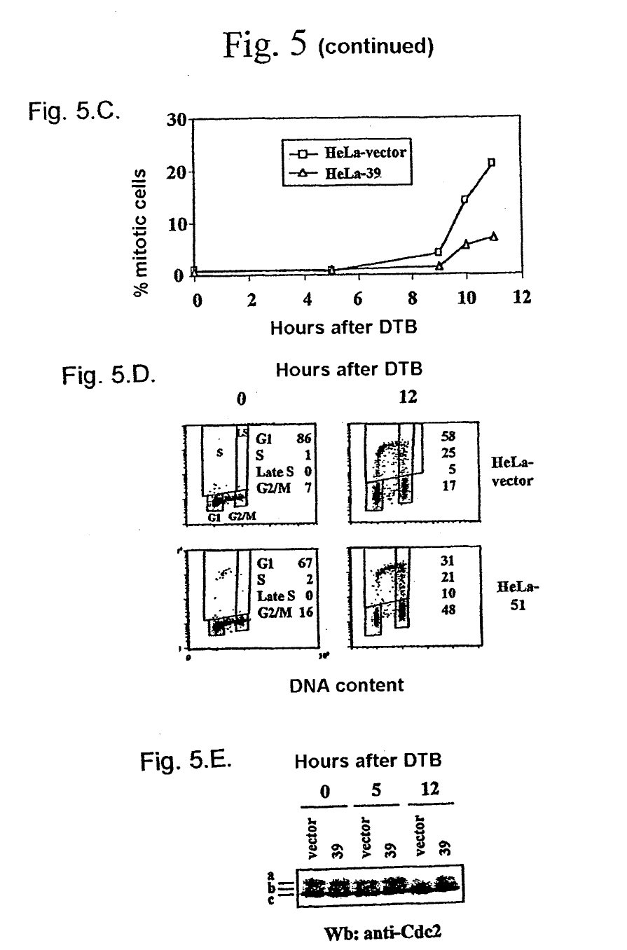

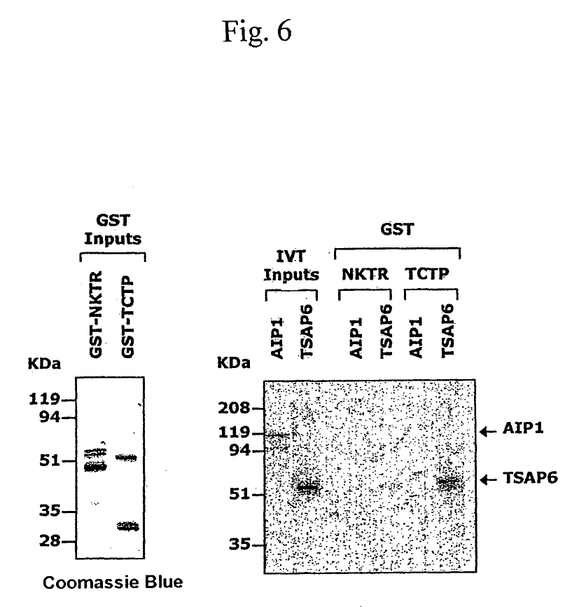

A. Western blotting analysis indicates that the endogenous protein levels for TSAP6 are decreased 24 h after induction by p53 in LTR6 cells overexpressing anti-TSAP6.

B. Overexpression of the TSAP6 antisense (as2) delays the apoptosis induced by activation with p53. The analysis of the evolution over time of LTR6 and LTR6-as2 according to temperature changes at 32° C.

C. Two-color flow cytometry analysis of the LTR6 and LTR6-as2 cells. Cells were labeled with annexin-V and propidium iodide (PI). The LTR6-as2 cells express the annexin-V and annexin-V/PI less compared to the LTR6 cells, indicating that the TSAP6 antisense prevents the p53-induced apoptosis.

D. LTR6-as2 and -as4 prevent p53-induced PARP cleavage. Total lysates from the experiments above were analyzed for PARP cleavage, a marker for apoptosis, with an anti-PARP antibody. Compared to the LTR6 cells, LTR6-as2 and -as4 show a marked decrease in PARP 8 hours after activation with p53.

FIG. 3: TSAP6 binds with Nix and Myt1 in vitro and in vivo

A. Analyses of a layer of yeast revealed that TSAP6 interacts with Nix and Myt1.

B. Interaction in vitro of either GST-Nix or GST-Myt1-150 with TSAP6 radiolabeled during in vitro translation (IVT). The GST-Nix or GST-Myt1-150 is incubated with TSAP6 transcribed/translated and radiolabeled in vitro. GST-NKTR and AIP1 labeled by IVT are also included as a negative control. These analyses revealed that GST-Nix and GST-Myt1-150 bound specifically with TSAP6. In addition, mapping experiments revealed that Nix and Myt1 interact with a fragment of TSAP6 composed of amino acids 1 to 316.

C. Interaction in vivo of Ha-TSAP6 with Flag-Nix (left panel) and Flag-Myt1-150 (right panel) in 293T cells. 293T cells were transfected with the indicated combination of plasmids and TSAP6 immunoprecipitated with an anti-HA antibody. A Nix and Myt1-150 interaction was revealed with an anti-Flag antibody.

D. Colocalization of GFP-TSAP6 and Flag-Myt1. Hela cells overexpressing GFP-TSAP6 (left panel) and Flag-Myt1 (middle panel) were analyzed with a confocal microscope for colocalization (right panel). This analysis indicates that TSAP6 and Myt1 are colocalized.

FIG. 4: TSAP6 cooperates with Nix to promote cell death

A. Hela 39 and 51 cells overexpressing HA-TSAP6. Western blotting analysis using an anti-HA on Hela cells in order to detect TSAP6.

B. Overexpression of TSAP6 promotes retarded cell growth and apoptosis. Analysis of cell growth (left panel). The Hela cells or the Hela cells expressing either a control vector or TSAP6 (39 and 51) were labeled with trypan blue and the cell viability was determined at the times indicated. Analysis of cell death (right panel): the cell death was determined by counting cells which had incorporated trypan blue.

C. Exacerbation of apoptosis in the Hela-51 cells overexpressing Nix. A Hela vector or Hela-51 cells were transfected with Flag constructs containing the negative control AIP1, Nix or the pro-apoptotic molecule Bid-t, which was used as a positive control. 24 hours after transfection, the cells were labeled with an anti-flag antibody so as to detect the overexpressed proteins, and with DAPI, a nuclear stain, and the cells were then counted. This analysis showed that, while AIP1 had a minimal effect on apoptosis in any cell line, overexpression of Nix greatly stimulated cell death in the cells overexpressing TSAP6. On the other hand, Bid-t promoted similar levels of apoptosis independently of the cell line.

D. Overexpression of Nix in Hela-51 greatly promotes PARP cleavage. Total lysates from the preceding experiments were analyzed by Western blotting using an anti-PARP antibody. This analysis shows that Nix and TSAP6 cooperate together to promote levels of PARP cleavage (below). The TSAP6 antisense prevents the Nix-induced PARP cleavage in 293T cells. 293T cells were transfected with the plasmids indicated and, 24 hours later, cell lysates were prepared and the PARP cleavage was analyzed by immunolabeling with an anti-PARP antibody. These data show that, while Nix alone promotes partial cleavage of PARP, the addition of a TSAP6 antisense completely blocks this effect.

E. Colocalization of GFP-TSAP6 and Nix after treatment with staurosporine. Hela cells transfected with TSAP6 (left panel) and Nix (middle panel) were analyzed with a confocal microscope 24 hours later. While colocalization of TSAP6 and Nix (right panel) was difficult to detect (-stauro), induction of apoptosis with staurosporine promoted coalescence of TSAP6 and Nix.

FIG. 5: Overexpression of TSAP6 promotes the delaying of the cell cycle in G2 by preventing dephosphoryation of cdc2

A. Double thymidine block analysis reveals that the Hela-51 cells contain an additional G2/M-population. A DTB procedure was carried out and a Hela vector and Hela-51 cells were analyzed in the times indicated with regard to their progression in the cell cycle. An analysis by flow cytometry was carried out on cells labeled with propidium iodide with the aim of revealing the various steps of the cell cycle. At. t=0, the Hela-51 cells showed an additional population of cells corresponding to G2/M. At t=12 hours, the Hela-51 cells contained a notable accumulation of these G2/M cells, at a time when the majority of Hela-vector cells are returning to stage G1.

B. H3 histone labeling at t=0 and t=12 hours after the release of DTB. The analysis by flow cytometry was carried out on cells labeled with an anti-H3 phosphohistone antibody, which labels cells in mitosis, and with propidium iodide. This analysis revealed that, while the majority of the Hela-51 cells exhibited a G2/M phenotype (approximately 60%) after 12 hours, only approximately 15% of the G2/M cells exhibited an H3⁺phenotype. This is in contrast with the Hela vector cells having G2/M, 20% of which are in mitosis at the same time.

C. Analysis of the mitotic index of the Hela-vector and Hela-51 cells. These results revealed that the Hela-51 cells have a delayed entry into mitosis compared to the Hela vector cells. In addition, these data confirm the results above according to which overexpression of TSAP6 delays rather than favors the progression of the cell cycle. The Hela cells were synchronized with DTB and, at the times indicated, the cells were fixed and stained with Giemsa-Wright stain with the aim of detecting the chromosomal condensations. At least 600 nuclei were counted for each time point.

D. Brdu labeling of the Hela-vector cells and of the Hela-51 cells. The Hela cells were labeled with Brdu, which detects the cells containing cells in the S phase, and with propidium iodide. Analysis by flow cytometry was carried out on the Brdu labeling. These results show that the late S-phase cells represent a small percentage of the cells in the G2/M phase for the two cell types, namely Hela-vector or Hela-51.

E. Cdc2 is hyperphosphorylated in the cells overexpressing TSAP6. Cell lysates were generated at the time points indicated after the release of DTB and immunolabeling analysis was carried out using an anti-cdc2 antibody in order to detect the various phosphorylated forms of cdc2. This analysis showed that, 12 hours after DTB release, the majority of cdc2 is dephosphorylated in the Hela-vector cells, indicating an active form. Surprisingly, on the other hand, cdc2 in the Hela-51 cells appears to be hyperphosphorylated at the same time.

FIG. 6: GST-TCTP interacts with TSAP6 in vitro

Interaction of GST-TCTP and of radiolabeled TSAP6. TSAP6 and the negative control AIP1 were generated by in vitro translation (IVT) in a rabbit reticulocyte lysate in the presence of ³⁵S-labeled methionine and ³⁵S-labeled cysteine. Equal amounts of radiolabeled products were incubated with either GST-TCTP, or GST-NKTR fusion proteins captured on the glutathione beads as a negative control. The radiolabeled proteins were eluted in a protein sample buffer, resolved under reducing conditions (0.7 mM of 2-β-mercaptoethanol) by sodium dodecyl sulfate polyacrylamide gel electrophoresis (SDS-PAGE) (10%), and visualized by autoradiography.

### EXAMPLES

### Example 1

Binding Between TSAP6 and the Proteins Chosen from SEQ ID No 1 to SEQ ID No 35 or SEQ ID No 44 or 46, or Encoded by a Nucleic Acid Chosen from SEQ ID No 36 to SEQ ID No 43 or SEQ ID No.45 or 47

The binding which exists between TSAP6 and each protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, was observed in a double-hybrid system derived from the system developed by Finley and Brent (Interaction trap cloning with yeast, 169-203, in DNA Cloning, Expression Systems: a practical Approach, 1995, Oxford Universal Press, Oxford), using TSAP6 as bait and a cDNA library as prey.

The TSAP6 protein was cloned into the plasmid pEG202, known to those skilled in the art for such an application (promoter 67-1511, lexA 1538-2227, ADH Ter 2209-2522, pBR remnants 2540-2889, 2μ ori 2890-4785, YSCNFLP 4923-5729, HIS3 7190-5699, TYIB 7243-7707, RAF_part 7635-7976, backbone pBR 7995-10166, bla 8131-8988).

The cDNAs of the library are cloned into the plasmid pJG4-5, also well known to those skilled in the art (promoter GAL 1-528, fusion cassette 528-849, ADH Ter 867-1315, 2μ ori 1371-3365, TRP1 3365-4250, backbone pUC 4264-6422, Ap 4412-5274).

The reporter plasmid pSH18-34, also known to those skilled in the art, is also used. This plasmid is in particular available from Invitrogen, under the reference number V611-20, and also already transformed into the strain EGY48 (also called RFY 231), from the same supplier (reference strain alone: C835-00, transformed with pSH18-34: C836-00).

The binding was demonstrated in the yeast stain RFY 231 (described in Finley Jr. et al, 1998, Proc Natl Acad Sci USA, 95, 14266-71). This yeast strain has the genotype (MATα trp1Δ::hisG his3 ura3-1 leu2::3Lexop-LEU2), and is derived from EGY48 (Guris et al., 1993, Cell, 75, 791-803).

The reporter gene was the LacZ gene.

The study is carried out on a medium containing galactose and not containing leucine, and the presence of colored colonies is studied on these dishes.

It is thus possible to show the binding, in this system, between TSAP6 and the proteins chosen from SEQ ID No 1 to SEQ ID No 35 or SEQ ID No 44 or 46, or encoded by a nucleic acid chosen from SEQ ID No 36 to SEQ ID No 43 or SEQ ID No 45 or 47.

### Example 2

Protocol for the Expression of the GST Fusion Proteins

To obtain GST fusion proteins, the following protocol can be followed:

Preparation of a preculture from an isolated colony of BL21 (DE3), transformed with the plasmid pGEX-6P-1 or pGEX-P-1, in an SB medium with 100 μg/ml of ampicillin, at 37° C.

The plasmids are available from Amersham Pharmacia Biotech AB.

The proteins are the human proteins encoded by the complementary cDNAs (SEQ ID-No 1 to SEQ ID No 7).

The following day, 250 ml of SB+Amp are inoculated with 5 ml of preculture.

Growth takes place at 28° C. or 37° C., depending on the toxicity of the proteins for the host bacteria, until an optical density of between 0.5 and 0.7 is reached. 0.1 mM IPTG is added to induce the protein synthesis.

Growth takes place at 28° C. or 37° C. for 1 h or 1 h 30.

Centrifugation is carried out at 3000 rpm for 10 min (1800 g, 4° C.).

The precipitate is resuspended in 10 ml of buffer A NP40 (1% NP40; 10 mM Tris pH 7.4; 150 mM NaCi; 1 mM EDTA; 10% glycerol; 1 mM DTT; 2 μg/ml Aprotinin; 2 μg/ml Leupeptin; 2 μg/ml Pepstatin; 1 mM AEBSF).

Sonication is carried out 3 times for 15 s at power 50, on ice.

Centrifugation is carried out at 12000 rpm for 10 min (18000 g, 4° C.).

The supernatant is kept at −80° C.

### Example 3

Protocol to be Followed for Binding of the Fusion Proteins to the Sepharose-glutathion Beads

2 ml of supernatant are added to 200 μl of beads (prepared after 3 rinses in PBS, 1 rinse in the buffer A NP40, resuspension at 50% (weight by volume) in the buffer A NP40, centrifugation at 3000 rpm each time).

The Glutathione-sepharose 4B beads are available from Amersham Pharmacia Biotech AB, under the number 17.0756.01.

Gentle mixing is carried out for at least 1 hour at 4° C.

The beads are rinsed 3 times in the buffer A NP40 without protease inhibitor.

The beads are resuspended in 1 ml of buffer A NP40 with protease inhibitor.

For the analysis by SDS-PAGE electrophoresis, a 20 to 40 μl sample of the resuspended beads is taken, centrifugation is carried out for 5 min, the supernatant is discarded, the beads are resuspended in 10 μl of loading buffer X, and heating is carried out at 97° C. for 7 min. The gel is loaded and analyzed after staining with Coomassie blue, to standardize the amount of the fusion proteins to be used.

### Example 4

Protocol for the in vitro Translation of Proteins

The TNT Coupled Reticulocyte Lysate System kit from Promega is used with the T7 or T3 RNA polymerases, depending on the vector used to translate and express the proteins (for example T7 for TSAP6 1, T3 for the protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48). The kit is used according to the manufacturer's instructions (Reference L4610).

The proteins incorporate S³⁵-methionine (Amersham Pharmacia).

The products obtained in vitro are analyzed by SDS-PAGE electrophoresis.

After electrophoresis, the gel is placed in fixing buffer. (5% of methanol, 15% of acetic acid, 80% of water) for half an hour and the signal is amplified by immersing the gel in the Amplify product from Amersham Pharmacia (Ref: NAMP100).

A Kodak Biomax MR film is then exposed on the dried gel for a period of time ranging from one hour to one week, and then developed.

### Example 5

Protocol for Coprecipitation of the Proteins

30 μl of the sepharose-glutathione beads coupled to the GST fusion proteins, after standardization of the amounts, are rinsed in buffer B (1% NP40, 50 mM Tris-HCL, 150 mM NaCl, 2 μl/ml leupeptin, 1% aprotinin, 1 mM ABESF). 5 to 10 μl of the in vitro translation product as obtained in example 3 are then added, depending on the amount of the product observed by autoradiography.

After contact overnight, the beads are rinsed 10 times with buffer A NP40, without antiproteases.

Analysis is carried out by SDS-PAGE and autoradiography.

It is thus possible to show the binding between the TSAP6 proteins and said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48.

### Example 6

Protocol for Screening Compounds which Interfere with the Binding Between TSAP6 and one of the Proteins from SEQ ID No 1 to SEQ ID No 35 or SEQ ID No 44, 45 or 47, or Encoded by a Nucleic Acid Chosen from SEQ ID No 36 to SEQ ID No 43 or SEQ ID No 46 or 48

The assays described in examples 2 to 5 are carried out, adding the compounds intended to be studied in the step of example 5, and the results obtained when the compounds are not added are compared.

### Materials and Methods

Expression and Purification of the GST Fusion Proteins

A preculture of TCTP pGEX-6P-1 or of NKTR in SB+100 μg/ml ampicillin was prepared, from a single colony, overnight at 37° C.

In the morning, 250 ml of LB+Amp were inoculated with 5 ml of this preculture.

Growth is carried out at 37° C. until the OD reaches 0.5 to 1 (approximately 3 hours).

0.1 mM of IPTG is added.

Growth is carried out for a further 1 h 30.

Centrifugation is carried out at 3000 rpm for 10 min.

The pellet is resuspended in 10 of a lysis buffer A NP40, in such a way as to avoid bubbles:

1% NP40*

10 mM Tris pH 7.4*

150 mM NaCl*

1 mM EDTA*

10% glycerol*

1 mM DTT

2 μg/ml Aprotinin

2 μg/ml Leupeptin

2 μg/ml Pepstatin

1 mM AEBSF.

Sonication is carried out 3 times for 15 seconds in ice, every 15 seconds, at power 40.

Centrifugation is carried out at 12000 rpm=18000 g (1510R centrifuge) at 40° C. for 30 min.

The supernatant is conserved with care at −80° C. if necessary.

Binding to the Agarose-glutathione Beads

2 ml of supernatant are added to 200 μl of beads (they should be prepared with 3 rinses with PBS, 1 rinse with the NP40 buffer and protease inhibitors, resuspended at 50% in this NP40 buffer, with centrifugation at 1500 rpm=900 g (1510R centrifuge) at 4° C., each time)

Gentle agitation is carried out for at least 1 hour at 4° C.

The beads are rinsed 3 times in buffer A NP40 without protease inhibitor in 10 ml of NP40 buffer each time (50 volumes).

200 μl of beads are resuspended in 200 μl of NP40 buffer with protease inhibitors, so as to obtain a 50% solution.

Analysis of the gel: 20 μl are centrifuged for 5 minutes, the supernatant is removed, the beads are resuspended in 10 μl, twice, in a sample buffer, and heating is carried out at 97° C. for 7 min. The gel is loaded in order to standardize an amount of GST fusion proteins. Novex 10% NuPAGE gels were used with MES buffer to obtain good resolution.

In vitro translation

The “TNT Coupled Reticulocyte Lysate System” kit from Promega was used with T7 polymerase. The standard protocol was followed.

The proteins were radiolabeled with ³⁵S methionine or ³⁵S methionine, cysteine from Amersham Pharmacia.

| | | | | | | | | |
|---|---|---|---|---|---|---|---|---|
| Total reaction volumes | 50 | μl | 100 | μl | 150 | μl | 200 | μl |
| Red lysate (μl) | 25 | | 50 | | 75 | | 100 | |
| TNT buffer (μl) | 2 | | 4 | | 6 | | 8 | |
| AA mix-Met or -Met and | 2 | | 4 | | 6 | | 8 | |
| -Cys (μl) | | | | | | | | |
| RNAsin (μl) | 1 | | 2 | | 3 | | 4 | |
| T7 RNA polymerase (μl) | 1 | | 2 | | 3 | | 4 | |
| 35S Met or 35S Met, Cys | 3 | | 5 | | 7 | | 9 | |
| (μl) | | | | | | | | |
| H20 (μl) | 15 | | 32 | | 49 | | 66 | |
| DNA pl | 0.5 | μg | 1 | μg | 1.5 | μg | 2 | μg |

The mixture was prepared in ice. The radioactivity was added at the end.

The reaction was carried out at 30° C. for 1 h 30.

During the in vitro translation, the beads were prepared: the amount of each fusion protein bound to agarose-glutathion beads was calculated. In each case, solid beads were added so as to have a total volume of 30 μl of beads per interaction, with the aim of being able to see them at the bottom of the tube. The beads were rinsed 3 times in 1 ml of buffer B: 1% NP40, 50 mM Tris-HCl, 150 mM NaCl, 2 μg/ml Leupeptin, 1% Aprotinin, 1 mM AEBSF.

An aliquot of 30 μl of beads (60 μl of a 50% solution) was placed in Eppendorf tubes. 50 μl of buffer B NP40+inhibitors was added to each tube so as to have a significant volume during the interaction. The positive and negative controls were included. It is advisable to avoid GST alone as a negative control since it is too viscous.

As soon as the in vitro translation has taken place, 3 to 30 μl of IVT (depending on the desired standardization) are added to each 30 μl of beads.

2X SB containing 0.7 mM of beta-mercapto ethanol is added to the remainder of the IVT in order to analyze the gel. The samples are heated at 80° C. for 5 min before being loaded.

After overnight contact at 4° C. or 1 hour at ambient temperature, the beads are rinsed 10 times in buffer A or B (if considerable stringency is acceptable) (10×1.5 ml) at 4° C. After rinsing, the beads are covered with 10 ml of buffer to which are added 30 μl of 2 times SB containing 0.7 mM of beta-mercapto ethanol and heated at 80° C. for 5 min before the analysis by SDS-PAGE. 12 to 20 μl of the samples can be loaded into each well. After electrophoresis, the gel is soaked in a fixing buffer (5% of methanol, 15% of acetic acid, 80% of water) for 30 min and the signal is amplified by immersing the gel in the Amplify product from Amersham Pharmacia.

A Kodak Biomax MR film is then exposed on the dry gel for 1 hour to 1 week and developed.

1. A method for identifying a compound which inhibits the binding of TSAP6 to one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, having the steps of:
a) bringing said compound into contact with a system for determining, in vitro, the binding between TSAP6 and one of the proteins chosen from SEQ ID No 1 to SEQ ID No 35 or SEQ ID No 44, 45 or 47 or encoded by a nucleic acid chosen from SEQ ID No 36 to SEQ ID No 43 or SEQ ID No 46 or 48;
b) identifying the decrease in and/or the inhibition of the binding between TSAP6 and said protein defined in a).

2. A method for identifying a compound which makes it possible to increase tumor reversion and/or cell death (apoptosis), characterized in that it has the steps of:
a) bringing compounds into contact with a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;
b) identifying the compounds which induce the decrease in and/or the inhibition of the binding between TSAP6 and said protein defined in a);
c) bringing the compounds identified in step b) into contact in a system for measuring the phenomena of apoptosis and/or of tumor reversion;
d) identifying the increase in tumor reversion and/or in cell death (apoptosis) in said model by comparison with a control model with which said compound has not been brought into contact.

3. A method for identifying a compound for decreasing and/or inhibiting tumor reversion and/or cell death (apoptosis), having the steps of:
a) bringing compounds into contact with a system for determining, in vitro, the binding between TSAP6 and one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;
b) identifying the compounds which induce the decrease in and/or the inhibition of the binding between TSAP6 and said protein defined in a);
c) bringing the compounds identified in step b) into contact in a system for measuring the phenomena of apoptosis and/or of tumor reversion;
d) identifying the decrease in and/or the inhibition of tumor reversion and/or cell death (apoptosis) in said model by comparison with a control model with which said compound has not been brought into contact.

4. A method for identifying regions of TSAP6 which are involved in the binding with one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, comprising the steps of:
a) bringing peptides derived from the TSAP6 protein into contact in a system for determining, in vitro, the binding between TSAP6 and said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48;
b) identifying the peptides which lead to the decrease in the binding between TSAP6 and said protein chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, in said system.

5. A method for identifying a product having an activity of increasing tumor reversion and/or apoptosis, characterized in that it comprises the steps of:
a) implementing a method as claimed in either of claims 1 and 2,
b) modifying the product selected in step a), in particular by grafting residues onto the chemical backbone,
c) testing the product modified in step b) in in vitro and/or in vivo methods on relevant models of tumor reversion and/or apoptosis,
d) identifying the product which makes it possible to obtain an activity of tumor reversion and/or apoptosis greater than the activity obtained for the product selected in step a).

6. A method for identifying a product having an activity of decreasing and/or inhibiting tumor reversion and/or apoptosis, characterized in that it comprises the steps of:
a) implementing a method as claimed in either of claims 1 and 3,
b) modifying the product selected in step a), in particular by grafting residues onto the chemical backbone,
c) testing the product modified in step b) in in vitro and/or in vivo methods on relevant models of tumor reversion and/or apoptosis,
d) identifying the product which makes it possible to obtain an activity of tumor reversion and/or apoptosis which is decreased compared to the activity obtained for the product selected in step a).

7. The use of a compound identified using a method as claimed in one of claims 1, 2, 4 and 5, having an activity of increasing tumor reversion and/or apoptosis, for preparing a medicinal product intended for the treatment of cancer.

8. The use of a compound identified using a method as claimed in one of claims 1, 3, 4 and 6, having an activity of decreasing and/or inhibiting tumor reversion and/or apoptosis, for preparing a medicinal product intended for the treatment of a neurodegenerative disease.

9. A peptide sequence corresponding to a region of TSAP6 which can be identified using a method as claimed in claim 4.

10. A nucleotide sequence encoding a peptide sequence as claimed in claim 9.

11. The peptide sequence as claimed in claim 9, or the nucleotide sequence as claimed in claim 10, as a medicinal product.

12. The use of a peptide sequence as claimed in claim 9, or of a nucleotide sequence as claimed in claim 10, for preparing a medicinal product.

13. A complex consisting of a TSAP6 protein and of one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID-No 46 or 48.

14. A method for inhibiting the binding of TSAP6 to one of the proteins chosen from SEQ ID No 1 to SEQ ID No 35 or SEQ ID No 44, 45 or 47, or encoded by a nucleic acid chosen from SEQ ID No 36 to SEQ ID No 43 or SEQ ID No 46 or 48, in a cell in vitro, comprising the step of:
a) bringing said cell into contact with a compound identified using a method as claimed in claim 1.
